SEQUENCE LISTING (2 sequences)

SEQ ID NO: 1 - DNA, 17 bp, ARTIFICIAL
  PRIMER
  misc_feature (9)..(9): n is a, c, g, or t
cctacgggnbgcascag

SEQ ID NO: 2 - DNA, 20 bp, ARTIFICIAL
  PRIMER
  misc_feature (8)..(8): n is a, c, g, or t
ggactacnvgggtwtctaat

(19) United States
(12) Patent Application Publication
(10) Pub. No.: US 2023/0227885 A1
(43) Pub. Date: Jul. 20, 2023

(54) MICROBIAL NICHE MAPPING

(71) Applicant: PHARMABIOME AG, SCHLIEREN (CH)

(72) Inventors: TOMAS DE WOUTERS, SCHLIEREN (CH); GABRIEL LEVENTHAL, SCHLIEREN (CH); PHILIPP ROGALLA VON BIEBERSTEIN, SCHLIEREN (CH); LAURA ANTHAMATTEN, SCHLIEREN (CH)

(21) Appl. No.: 18/007,116

(22) PCT Filed: Jul. 29, 2021
(86) PCT No.: PCT/EP2021/071235

§ 371 Date: Jan. 27, 2023

(30) Foreign Application Priority Data
Jul. 31, 2020 (EP) 20188833.6

(51) Int. Cl. C12Q 1/06; C12Q 1/689

(52) U.S. Cl. CPC C12Q 1/06; C12Q 1/689

(57) ABSTRACT

The invention concerns methods for mapping microbial niches and their uses.

### TECHNICAL FIELD

The present invention relates to the fields of biotechnology, microbiology and medicine and in particular to the mapping of microbial niches.

### BACKGROUND OF THE INVENTION

The microbiota is composed of more than a thousand different microbial species and because of its complexity and multitude of functions is sometimes described as “an additional organ”. It plays a beneficial role for the host by exerting many biological functions. For example, in the gut, the microbiota is crucial for nutrient absorption, maintenance of intestinal epithelium integrity, protection from pathogens, and homeostasis of immune responses. Similarly, in and on other body sites like the skin, microorganisms have essential roles like protecting the host from pathogens, maintaining health promoting micro-environmental conditions, as well as immunological functions.

The underlying cause of multiple microbiome-linked diseases is a dysbiosis of the microbiota, i.e. a detrimental change in microbiota composition. In the gut, such dysbiosis relates for instance to inflammatory bowel disease or ulcerative colitis, where the dysbiotic microbiome composition chronically induces colitis in the patient. Such deviations from a healthy microbiome are highly challenging to identify and describe by pure taxonomic analyses, because all individuals carry different specific microbial species that are functionally redundant or complementary and thereby de-couple taxonomic attribution from functional potential. Gene-based or motif-based functional analyses also fall short due to the still limited understanding of the genetic encoding of the multitude of functionalities in the intestine. More importantly there is even less understanding on the contextual expression of these specific genes limiting gene-based prediction to the description of possibilities rather than activities.

It is therefore an object of the present invention to provide a method to reliably identify functional deviations from a reference microbiome, such as a healthy microbiome, by mapping bacteria to niches within the habitat, i.e. niche mapping. The present invention can be used to establish a niche map for microbiota in a variety of environments, e.g. associated to different human and animal hosts, plants, or biotechnological use of microbiota, and thus identify functional deviations from healthy or optimal microbiota.

### SUMMARY OF THE INVENTION

The present invention relates to a niche map, to methods of building a niche map, and the use of the niche map as a drug development platform, diagnostic and personalized medicine or nutrition tool, as well as compositions developed by using a niche map. However, the utility of a niche map is not limited to the field of medicine. It is also relevant in other fields such as agriculture, farming, environment, and food industry.

Metabolic interactions are key drivers of microbiome composition. As a result, therapeutic interventions must not target just a single bacterium but the interactions that define the composition and metabolic activity of a microbiome and thereby the effect on their host or environment. Because microbial interactions depend (1) on the specific bacterial genotypes that are interacting and (2) on nutrients and physicochemical parameters at the moment of the interaction, meaningful computational predictions of microbiome metabolic activity are strongly limited to the comparatively small fraction of bacteria that have been isolated to date. To characterize the metabolic niches of bacteria more broadly, the inventors developed a ‘niche mapping’ platform that does not focus on single bacteria but rather on the set of bacteria specific to a niche of a microbiome, such as the microbiome of the human intestine.

A niche is defined as a habitat with defined niche conditions such as substrates as the growth-promoting energy source and environmental physicochemical parameters, such as but not limited to pH conditions, temperature, Redox conditions, presence or absence of secondary metabolites or specific co-factor. Such physicochemical parameters affect exertion of the previously described functions and influences the metabolic abilities of bacteria. Enrichment experiments using niche conditions, such as defined substrates and defined physicochemical parameters allow the identification of bacterial strains with the highest competitive advantage in a specific niche. By subtracting niche-unspecific growth, i.e. strains that grow under reference conditions, bacterial strains most competitive for a microbiome niche are found. By iterating this process for different niche conditions, a niche map can be established that describes the most advantageous conditions for competitive growth of the multitude of bacteria in the ecosystem and thus the main drivers defining composition of a specific microbiome.

The niche map can be used as a platform for analyzing dysbiosis in a subject or group of subjects. For instance, the niche map can be built for a specific patient or a patient group and compared to the niche map of a healthy population. Thus, the niche map could be an important tool for developing a suitable treatment for a patient or a patient group.

The invention particularly concerns a method for establishing a microbiome niche map comprising the steps of:
a. Providing a microbiome sample distributed into at least two microbiome test samples,
b. Growing a first microbiome test sample of step (a) in a reference niche condition,
c. Determining an absolute or relative abundance of individual microbe population in the first microbiome test sample at the end of step (b),
d. Growing a second microbiome test sample of step (a) in a niche condition that differs in at least one parameter from the reference niche condition,
e. Determining an absolute or relative abundance of individual microbe population in the second microbiome test sample at the end of step (d),
f. Determining microbe population differentially enriched between the first and the second test sample by subtracting the absolute or relative abundance of an individual microbe population of step c) from the absolute or relative abundance of the same individual microbe population of step e),
g. Optionally, repeating steps (b) to (f) for at least one other niche condition, and
h. Attributing the enriched microbe population of step (f) or (g) to a specific niche condition.

In a first embodiment, the method uses the absolute abondance.

In particular, the absolute abundance of the individual microbe population is determined by (i) determining the total microbial growth at the end of the growing step (b) or (d), and (ii) determining the relative abundance of the individual microbe population grown at the end of the growing step (b) or (d), respectively.

Preferably, the absolute abundance of the individual microbe population is determined by (i) determining the total increase of optical density or microbial DNA at the end of the growing step (b) or (d), and (ii) sequencing the total microbial DNA at the end of the growing step (b) or (d), respectively.

In a second embodiment, the method uses the relative abundance.

Particularly, in step (a), the method further comprises one or several dilutions of the microbiome sample in a suitable dilution agent, preferably to obtain a dilution by a factor of at least 10, preferably by a factor comprised between 10¹ and 10¹².

In some aspect, in step (a), the method further comprises a pre-treatment step of the microbiome sample, in particular using heat, pH stress, bleach or ethanol.

In a certain aspect, the step (g) is repeated several times, preferably at least two times, with each time a different niche condition or parameter.

The niche condition parameter can be selected from the group consisting of substrate, pH, Oxidation-Reduction Potential (Redox), temperature, humidity, pressure, cultivation method, incubation time, inhibitory factors and promoting growth factors.

Preferably the niche condition parameter is a niche substrate selected from the group consisting of carbohydrate, fiber, protein, gas, organic molecules of animal, fungi or plant origin, phenols, hormones, nucleotides and amino acids.

Particularly, the niche substrate is selected from the group consisting of polysaccharides, non-starch polysaccharides (NSP), resistant starch (RS) and oligosaccharides (RO), preferably in the group consisting of Cellulose, Hemicellulose, Guar Gum, Gum Arabic, Lignin, Fructan (long chain length), Inulin (long chain length); Arabinogalactan, Arabinoxylan, B-Glucan, Galactomannan, Glucomannan, Xyloglucan, Xylan, Amylo-pectin, Pectin, Starch (Type 1 to Type 9), Resistant starch (Type 1 to 3), Resistant dextrins, Arabinose, Fructose, Glucose, Galactose, Galacturonic Acid, Xylose, Lactose, Lactulose, Maltose, Sucrose, Galactooligosaccharides (GOS), Fructooligosaccharides (FOS), Xylooligosaccharides (XOS), Mannans, Pectin, Inulin, Polydextrose, Fungal Carbohydrates, Yeast carbohydrates, Chitin, Pullulan, Mucus, type I-type 4 mucus, N-acetyl-galactosamine, N-acetyl-glucosamine, Galactose, Fucose, human milk oligosaccharides, Siliac acid, N-Acetylneuraminic acid, Cell-surface glycans, GABA, surface glycosylation, Hormones, Cholesterol, Bile acids, yeast extract, casein, meat extract, blood, brain heart infusion broth, rumen fluid, sterile fecal suspension, amino acids, nucleic acids, biogenic amines, fetal calf serum, Acetate, Lactate, Formate, Succinate, H2, CO2, Ethanol, 1,2-Propanediol, and any combination thereof.

The microbiome sample can be provided from an intestinal microbiome, a mouth or nasal microbiome, a vaginal microbiome, a skin microbiome, a waste-treatment microbiome, a food microbiome, a microbiome used for food fermentation, oil spills microbiome, water microbiome such as a microbiome from lakes and waters, a soil microbiome or a plant-associated microbiome.

In a particular aspect, the method is carried out for microbiome samples from different subjects or population of subjects.

The invention also relates to the use of the method according to the invention for identifying a pattern of enriched individual microbe populations associated to a condition, the condition being (i) a dysbiosis, (ii) a presence of or a susceptibility to develop a disease, (iii) a susceptibility of a subject to be a responder or a non-responder to a treatment, for instance a treatment with a drug or diet, (iv) a susceptibility of a subject to present or not side effects to a treatment with a drug, or (v) a good health.

The invention also concerns a method for developing a probiotic composition susceptible to benefit to a patient suffering from dysbiosis, wherein the method comprises:
(a) determining the niche map of the subject microbiome by the method according to the invention,
(b) comparing the microbiome niche map of the subject to a microbiome niche map of a healthy subject or a healthy subject population,
(c) determining under-representation and/or over-representation of niches in the subject microbiome compared to the niche map of a healthy subject or a healthy subject population, and
(d) developing a composition comprising one or more bacterial strains able to colonize an underrepresented niche and optionally, one or more bacterial strains as stabilizing agents.

The invention also relates to a method for predicting the response of a subject to a treatment with a drug or a diet, wherein the method comprises:
(a) determining the niche map of the subject microbiome by the method according to the invention,
(b) comparing the microbiome niche map of the subject to a microbiome niche map of reference, the reference being a microbiome niche map of a good-responder population to such treatment or diet and/or a microbiome niche map of a non-responder population to such treatment or diet, and
(c) determining under-representation and/or over-representation of niches in the subject microbiome compared to the niche map of reference, wherein under-representation or over-representation of niches in the subject microbiome being indicative of the response of the subject to such treatment or diet.

Preferably, the microbiome sample is from a mammal, preferably a human.

### DESCRIPTIONS OF THE FIGURES

FIG. 1: Example of the steps of the Niche Mapping method according to an embodiment of the invention.

FIG. 2: Example of a Niche Mapping in three dimensions.

FIG. 3: Example the mapping of the succinate-utilizing niche. Bacteria that are mapped to the succinate-utilizing niche using niche mapping correspond to known succinate utilizing strains. Pfa: Phascolarctobacterium faecium, Dsu: Dialister succinatiphilus. Succinate utilization is a phylogenetically highly conserved trait and thus a strong validation of the presented approach. Three distinct microbiome samples were enriched in a basal medium with succinate as a niche substrate. The enrichment scores of all bacterial strains identified from DNA sequencing were calculated and those strains with an enrichment score in the top 97.5% were considered putative members of the succinate niche group. Each microbiome sample yielded one strain (Pfa=Phascolarctobacterium faecium; Dsu=Dialister succinatiphilus), both of which are known and confirmed succinate utilizers.

FIG. 4: The Lactate-utilizing niche yields altered compositions based on the presented pH condition.

### DETAILED DESCRIPTION OF THE INVENTION

### Niche Mapping

The invention concerns a niche mapping method based on enrichment experiments.

The invention particularly concerns a method for establishing a microbiome niche map comprising the steps of:
a. Providing a microbiome sample distributed into at least two microbiome test samples,
b. Growing a first microbiome test sample of step (a) in a reference niche condition,
c. Determining an absolute or relative abundance of individual microbe population in the first microbiome test sample at the end of step (b),
d. Growing a second microbiome test sample of step (a) in a niche condition that differs in at least one parameter from the reference niche condition,
e. Determining an absolute or relative abundance of individual microbe population in the second microbiome test sample at the end of step (d),
f. Determining microbe population differentially enriched between the first and the second test sample by subtracting the absolute or relative abundance of an individual microbe population of step c) from the absolute abundance of the same individual microbe population of step e),
g. Optionally, repeating steps (b) to (f) for at least one other niche conditions, and
h. Attributing the enriched microbe population of step (f) to a specific niche condition.

Preferably, the method is an in vitro method.

In a one aspect, the method uses the absolute abundance.

In an alternative aspect, the method uses the relative abundance.

The different steps of the method are more particularly described hereafter.

In one embodiment, the method comprises a step (g) consisting of repeating steps (b) to (f) for at least one other niche conditions.

### Microbiome Sample

The method disclosed herein comprises a step of providing a microbiome sample.

By “microbiome sample” it is meant a small part or quantity or a subset of a microbiome population, in particular a sample of the interacting microorganisms that form a microbiome. A microbiome sample may be random or nonrandom; representative or nonrepresentative of a microbiome. Preferably, the “microbiome sample” is intended to recapitulate the features of the whole microbiome of interest.

Then, the method according to the invention may further comprise a step of microbiome sampling, for example by sampling microbiome in water, soil or subjects.

In one embodiment, the microbiome sample is provided from an intestinal microbiome, a mouth or nasal microbiome, a lung microbiome, a vaginal microbiome, a skin microbiome, a waste-treatment microbiome, food microbiome, oil spills microbiome, water microbiome such as from lakes and waters, a soil microbiome, a plant-associated microbiome or a microbiome used for anaerobic food fermentation. For example, the microbiome sample can be provided through smear or fecal material. In one embodiment, the microbiome sample is a fresh fecal material.

Particularly, the microbiome sample can be from a microbiome that occur in a habitat such as on or in the human body, such as but not limited to a sample of microbiome of the skin, lung, intestine, mouth, female reproductive tract. A microbiome sample can also be a sample of interacting microbiomes that live on or in an animal. A microbiome sample can also be a soil sample or a sewage sample.

In a particular embodiment, the microbiome sample is provided from a subject, preferably an animal or mammal, in particular a human.

The method can be carried out for several microbiomes, for example microbiome samples from the same subjects, from different subjects or from population of subjects.

The method can particularly be carried out for two or at least two microbiome test samples. Preferably, the method can be carried out with 2, 3, 4, 5, 6, 7, 8, 9, 10, 11, 12, 13, 14, 15, 16, 17, 18, 19, 20, 30, 40, 50, 100, 500 microbiome samples or more, preferably at the same time. In one embodiment, at least 2, 3, 4, 5, 6, 7, 8, 9, 10, 11, 12, 13, 14, 15, 16, 17, 18, 19, 20, 30, 40, 50, 100, 500 niche parameters or more can be tested in the same experiment. In one embodiment, the method allows the test up to 1000 or 10 000 microbiome test samples at the same time (i.e., in parallel).

In one embodiment, the microbiome sample is prepared prior to its use in the method. Then, the method may comprise a step of microbiome sample preparation and/or pre-treatment.

In one embodiment, the microbiome sample is diluted prior to its use. Inoculation using diluted bacterial cultures are known in the field.

Then, in one embodiment, in step (a), the method further comprises one or several dilutions of the microbiome sample in a suitable dilution agent, preferably to obtain a dilution by a factor of at least 10, preferably by a factor comprised between 10³ and 10¹², preferably between 10³ and 10⁹, even more preferably between 10⁷ and 10⁹.

In one embodiment, the dilution is of 2logs compared to the initial microbiome sample. For example, a microbiome sample from the gut comprises generally a density of 10¹¹ bacteria. Then, a 2logs dilution represents a density of 10⁹ bacteria in the diluted microbiome sample.

In particular, low dilutions allow the niche mapping of dominant bacteria, fast growers and bacteria showing high affinity for cultivation and niche conditions. High dilutions allow the niche mapping of sub-dominant bacteria, slow growers and bacteria showing low affinity for cultivation and niche conditions.

In particular, the microbiome sample can be diluted in any suitable dilution agent. A “suitable dilution agent” is known to the person skilled in the art, and is a liquid composition that does not interfere with the microbiome nor the bacteria of the microbiome sample, e.g., distilled water or PBS.

In one embodiment, the microbiome sample is diluted in a suitable agent and optionally treated with further agents such as e.g., NaCl, phosphate buffer, acidic pH buffer, basic pH buffer, minerals, reducing agents such as cysteine, surfactants such as Tween20, bleach or ethanol, bacteriostatic or antibiotic agents or any combination thereof.

In one embodiment, the microbiome sample is previously treated by applying a stress, such as heat, inactivation with ethanol or bleach, inactivation by oxygen exposure, limited oxygen exposure, low pH buffer, high pH buffer, addition of bacteriostatic or antibiotic agents or any combination thereof.

### Basal Medium

A basal medium is used in the method of the present invention for the step of growing. It provides conditions for bacterial growth.

The terms “basal medium”, “dispersing medium”, “cultivation medium” and “culture medium” are used interchangeably herein and refer to a liquid or solid medium in which the bacterial strains are inoculated and/or cultivated. It is a medium that does not favor any specific bacterial group.

The basal medium particularly ensures that bacteria remain as viable live bacteria. Further, the basal medium comprises nutrients and allows growth of bacteria from the microbiome sample. For instance, the basal medium is able to maintain at least 50% of the bacterial diversity of the microbiome sample. Preferably, the basal medium is able to maintain at least 50, 55, 60, 65, 70, 75, 80, 85 or 90% of the bacterial diversity of the microbiome sample.

A broad range of solid or liquid basal media are known and may be used in the context of the present invention.

Suitable media include liquid media and solid supports. Liquid media generally comprise water and may thus also be termed aqueous media. Such liquid media may comprise a culture medium, a cryoprotective medium and/or a gel forming medium. Solid media may comprise a polymeric support.

Cultivation methods and in particular also the handling and cultivating of bacteria are known and e.g., described by the Leibniz Institute DSMZ—German Collection of Microorganisms and Cell cultures available from the internet https://www.dsmz.de/catalogues/catalogue-microorganisms/culture-technology.html

In one embodiment, the basal medium comprises fibers, intermediate substrates, arabinogalactan, soluble starch, pectin, resistant starch, casein, yeast extract, meat extract, mineral, SCFA, vitamin, resazurin, hemin, sodium bicarbonate, L-Cysteine hydrochloride monohydrate, potassium phosphate dibasic Trihydrate, Potassium dihydrogen phosphate, Sodium chloride, Ammonium sulfate, Magnesium sulfate, Calcium chloride dihydrate, larch tree, potato, Citrus peel, corn, Casein acid hydrolysate from bovine milk and/or Resazurin Sodium Salt.

In one embodiment, the basal medium further comprises agar.

### Niche and Growing Conditions

The “niche condition” relies on different parameters specific for a niche, such as substrate, pH, Redox, inhibitors and any parameters that affects bacterial growth and survival. The “niche parameters” or “parameter of a niche condition” are any characteristic defining a niche, such as any physicochemical condition, and/or any substance, compound or molecule in the culture medium, such as non-exhaustively substrate, pH, Redox, temperature and the like.

In the method according to the invention, such a parameter relies either on the basal medium composition or preparation, or on the culture or growing steps of the microbiome sample.

For example, when the parameter is a chemical entity, e.g., substrate, then the basal medium comprises a particular chemical entity, e.g., a particular substrate. When the parameter is temperature, the microbiome sample is grown under a particular temperature or temperature range. The man skilled in this art knows how to define and apply a particular niche parameter to a microbiome sample. The man skilled in this art also knows how to assess or monitor such parameter, for example using pH meter, thermometer, barometer, hydrometer and the like.

In one embodiment, the niche condition is selected from the group consisting of chemical entity, substrate, pH, Oxidation-Reduction Potential (Redox), temperature, flow rate, humidity, pressure, radiation, retention time, incubation time, inhibitory factors, and promoting growth factors.

In one embodiment, the niche parameter is temperature. In particular, the temperature may be comprised between −25° C. and 125° C. The temperature parameter may be −25, −20, −15, −10, −5, 0, 5, 10, 15, 20, 25, 30, 35, 40, 45, 50, 55, 65, 70,75, 80, 85, 90, 95, 100, 105, 110, 115, 120 or 125° C. Preferably, the temperature is comprised between 18° C. and 45° C., even more preferably between 20° C. and 37° C.

In one embodiment, the niche parameter is pressure. In particular, the pressure may be comprised between 0.1 to 130 MPa.

In one embodiment, the niche parameter is Oxidation-Reduction Potential (Redox). Redox is defined as the relative ease with which a medium gains (reduction) or loses (oxidation) electrons. The Redox potential can be comprised between −250 mV and 500 mV, in particular 300 mV to 500 mV, −100 to 300 mV, −250 mV to 100 mV, or less than −200 mV.

Such Redox conditions can be linked to aerobic or anaerobic conditions. Thus, in one embodiment, the Redox potential is comprised between 300 mV and 500 mV for aerobic conditions, between −100 mV and 300 mV for microaerobic conditions, between −250 mV and 100 mV for anaerobic conditions, or less than −200 mV for strict anaerobic conditions.

In one embodiment, the niche condition parameter is pH. Then, the niche may comprise alkaline or acidic conditions, e.g., the basal medium has an alkaline, acidic or neutral pH. Preferably, the pH is comprised between 4 and 8, preferably between 4.5 and 7.5, even more preferably between 4.7 and 7.5. In one embodiment, the pH is comprised between 4.5 and 5, 5 and 5.5, 5.5 and 6, 6 and 6.5, 6.5 and 7, 7 and 7.5. Preferably, the pH is 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4 or 7.5. In one embodiment, the pH is 6.5.

In one embodiment, the parameter is desiccation, humidity or moisture content. In particular, the basal medium comprises 50%, 60%, 70%, 80%, 90%, 95%, 100% humidity.

In one embodiment, the parameter is the salinity. In particular, the salinity parameter may range from the marine environment (˜3-4% salinity), hot springs (up to 10.5% salinity), and to soda lakes (up to 37.1% salinity), and even salt inclusions (up to 49.7% salinity). Preferably, the basal medium comprises a salinity of between 0 and 35%. A wide range of different ions, including Na²⁺, Cl⁻, SO₄²⁻, Ca²⁺, and Mg²⁺ can contribute to total salinity in the basal medium.

In one embodiment, the basal medium can comprise a chemical entity. A chemical entity can be for instance an amino acid, peptide or protein, a nucleotide or a polynucleotide, any chemical compound such as a drug, drug candidate, or any compound to be evaluated, a lipid, a mono-, di-, tri-, oligo- or polysaccharides. The chemical entity can be of any type, such as a small molecule, a macromolecule, a gas or more complex substances such as bacteria, fungi, viruses or parasites. Macromolecules include, without limitation, peptides, proteins and nucleic acids. The chemical entity can be part of a library of substances, for example a library of small molecules or a library of macromolecules.

In one embodiment, the basal medium can comprise an inhibitor selected from the group consisting of plant produced bacterial inhibitor such as diabolin, curcumin, allicin or capsin; phenols such as humic acids, cinnamic acids and benzoic acid, anthocyans, catechins, pepsin, antibodies such as IgA and IgE; Immune cell release substances such as H2O2 or ROS, bile acids, oxygen, bacterial metabolites such as fatty acids, Short chain fatty acids (SCFA, such as acetate, butyrate, propionate), Branched chain fatty acids (BCFA, such as isobutyrate, valerate, isovalerate), Biogenic Amines such as cadaverine, putrescine, spermidine, and histamine, Organic acids such as citric acid, fumaric acids, lactate, succinate and formate, Alcohols such as ethanol, methanol; bacterial antimicrobials such as bacteriocins; hydrogen.

In one embodiment, the niche parameter is a niche substrate. Then, in such embodiment, the basal medium further comprises a particular substrate.

By “substrate” and “niche substrate” it is meant a compound that is used by bacteria to grow. The term “substrate” is known and encompasses “nutrients” and other components of a medium supporting proliferation of one or more bacterial strain. The term “nutrient” in this text particularly refers to a component of the basal medium that some bacterial strains are capable of metabolizing, i.e., nutrients that can be converted into metabolites or energy.

By “absence of a niche substrate” it is meant that the basal medium is lacking a particular niche substrate.

In one embodiment, the niche substrate is a carbon source, a nitrogen source or a sulfur source or any combination thereof.

In one embodiment, the niche substrate is selected from the group consisting of carbohydrate, fiber, protein, gas, organic molecules of animal, fungi or plant origin, phenols, hormones, nucleotides and amino acids.

Preferably, the niche substrate is selected from the group consisting of Plant Carbohydrates/Oligosaccharides, Monosaccharide, disaccharide, oligosaccharides, Fungal Carbohydrates, Host derived carbohydrates, Protein and amino acid sources and bacterial metabolites.

In one embodiment, the substrate is a carbon source, preferably selected from the group consisting of Plant Carbohydrates/Oligosaccharides, Monosaccharide, disaccharide, oligosaccharides, in particular Non-starch polysaccharides (NSP), Hemi-cellulose, Cellulose, Pectin and Starch polysaccharides or any combination thereof.

In one embodiment, the niche substrate comprises Fungal Carbohydrates or Host derived carbohydrates, such as Yeast carbohydrates, Chitin, Pullulan, Mucus, type I-type 4 mucus, N-acetyl-galactosamine, N-acetyl-glucosamine, Galactose, Fucose, human milk oligosaccharides, Siliac acid, N-Acetylneuraminic acid, Cell-surface glycans, GABA, surface glycosylation, Hormones, Cholesterol, Bile acids or any combination thereof.

In one embodiment, the niche substrate comprises at least one bacterial metabolite, preferably selected from the group consisting of Acetate, Lactate, Formate, Succinate, H₂, CO₂, Ethanol, 1,2-Propanediol, and any combination thereof.

In one embodiment, the niche substrate comprises at least one protein or amino acid, preferably selected from the group consisting of yeast extract, casein, meat extract, blood, brain heat infusion, amino acids, nucleic acids, biogenic amines, fetal calf serum and any combination thereof.

Particularly, the substrate is selected from the group consisting of Guar Gum, Gum Arabic, Lignin, Fructan (long chain length), Inulin (long chain length); Arabinogalactan, Arabinoxylan, B-Glucan, Galactomannan, Glucomannan, Xyloglucan, Xylan, Cellulose, Amylo-pectin, Pectin, Starch (Type 1 to Type 9), Resistant starch (Type 1 to 3), Resistant dextrins, Arabinose, Fructose, Glucose, Galactose, Galacturonic Acid, Xylose, Lactose, Lactulose, Maltose, Sucrose, Galactooligosaccharides (GOS), Fructooligosaccharides (FOS, short chain length, e.g. short form of inulin, fructan), Xylooligosaccharides (XOS), Polydextrose, Fungal Carbohydrates, Yeast carbohydrates, Chitin, Pullulan, Mucus, type I-type 4 mucus, N-acetyl-galactosamine, N-acetyl-glucosamine, Galactose, Fucose, human milk oligosaccharides, Siliac acid, N-Acetylneuraminic acid, Cell-surface glycans, GABA, surface glycosylation, Hormones, Cholesterol, Bile acids, yeast extract, casein, meat extract, blood, brain heat infusion, rumen fluid, sterile fecal suspension, amino acids, nucleic acids, biogenic amines, fetal calf serum, Acetate, Lactate, Formate, Succinate, H2, CO2, Ethanol, Propandiol and any combination thereof.

Preferably, the niche substrate is selected from the group consisting of polysaccharides, non-starch polysaccharides (NSP), resistant starch (RS) and oligosaccharides (RO), preferably in the group consisting of Fructooligosaccharides (FOS), Xylooligosaccharides (XOS), Galactooligosaccharides (GOS), Resistant dextrins, Polydextrose, Cellulose, Hemicellulose, Mannans, Pectin, Inulin and Fructans.

In one embodiment, the basal medium comprises a particular substrate, such as simple sugars carbon (glucose, galactose, maltose, lactose, sucrose, fructose, cellobiose), “fibers” (preferably dietary fibers such as pectin, arabinogalactan, beta-glucan, soluble starch, resistant starch, fructo-oligosacharides, galacto-oligosacharides, xylan, arabinoxylans, cellulose), proteins (preferably yeast extract, casein, skimmed milk, peptone), co-factors (short chain fatty acids, formate, lactate, succinate, hemin, FeSO4), vitamins (preferably biotin or D-(+)-Biotin (Vit. H), Cobalamin (Vit. B12), 4-aminobenzoic acid or p-aminobenzoic acid (PABA), folic acid (Vit. B11/B9), pyridoxamine hydrochloride (Vit. B6)), minerals (preferably sodium bicarbonate, potassium phosphate dibasic, potassium phosphate monobasic, sodium chloride, ammonium sulfate, magnesium sulfate, calcium chloride) and reducing agents (preferably cysteine, titanium(III)-citrate, yeast extract, sodium thioglycolate, dithiothreitol, sodium sulfide, hydrogen sulfite, ascorbate), guar gum, glycerol, potato starch, rice starch, pea starch, corn starch, wheat starch, inulin, succinate, formate, lactate, iron sulfate, tryptone, fucose, acetate, mucus, trehalose, mannitol, polysorbate and any combination thereof.

In one embodiment, the niche substrate is a fiber. The term “fiber” is known and denotes in this text any carbohydrate polymer with more than ten monomeric units and refers in particular to plant fibers, modified plant fibers and dietary fibers. Fibers are generally not completely hydrolyzed in the small intestine of humans. Exemplary fibers include e.g., waxes, lignin, polysaccharides, such e.g., as cellulose, starch, resistant starch and pectin.

In one embodiment, the substrate comprises intermediate metabolites. The term “intermediate metabolite” denotes the metabolites produced by members of the microbiota that are used as energy source by other members of the microbiota. Such intermediate metabolites in particular may include degradation products from fibers, proteins or other organic compounds, but also formate, lactate and succinate that are typical intermediate products of known metabolic pathways. They are usually not found in healthy individuals. In particular, they are typically not enriched in the feces of a healthy individual.

In one embodiment, the niche substrate is one or more of lactate, succinate and formate.

In one embodiment, the parameter is lactate and/or succinate.

In one embodiment, the niche substrate comprises acids such as acetate, propionate and/or valerate.

In one embodiment, the niche substrate may comprise co-factors for growth such as vitamins, minerals and/or acids and/or growth enhancer.

In one embodiment the niche substrate comprises vitamins, in particular selected from the group consisting of Thiamine (Vit. B1 HCl), (−)-Riboflavin (Vit. B2), Nicotinic acid (Vit. B3), Pyridoxine-HCl (Vit. B6), Folic acid (Vit. B9), Cyanocobalamin (Vit. B12), Biotine (Vit. H), 4-Aminobenzoic acid (PABA), Phylloquinone (Vit. K1), Menadione (Vit. K3), Pantotenate (Vit. B5), Lipoic acid and any combination thereof.

In one embodiment, the niche parameter is a mineral, so that the niche substrate comprises a mineral, in particular such as KH2PO4, NH4Cl, KCl, CaCl2*2H2O, NaCl, MgCl2*6H2O (hexahydrat), NaSO4 (sodium sulfate), Casamino acids (casein hydrolysat) peptone from casein, Na2WO4, NazSeO3, (NH4)2SO4, and MgSO4 or any combination thereof.

In one embodiment, the parameter is the incubation time, culturation time or window of growth of the microbiome sample on the basal medium. Particularly, the incubation time is comprised between 5 hours and one month. In particular, the incubation time is of 8 h, 12 h, 16 h, 18 h, 24 h, 48 h, 72 h, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, or 30 days.

In one embodiment, the parameter is aerobiosis or anaerobiosis.

In one embodiment, the parameter is the oxygen content. Particularly, the oxygen content can be 0%, 0.5%, 1%, 5%, 10%, 15%, 20%, 25%, 30%, 35%, 40%, 45%, 50%, 55%, 65%, 70%, 75%, 80%, 85%, 90%, 95%, or 100%. This oxygen content can be defined by the man skilled in the art to create aerobic or anaerobic conditions.

In one embodiment, the parameter is the CO2 content. Particularly, the CO2 content can be 0%, 0.5%, 1%, 5%, 10%, 15%, 20%, 25%, 30%, 35%, 40%, 45%, 50%, 55%, 65%, 70%, 75%, 80%, 85%, 90%, 95%, or 100%.

In one embodiment, the parameter is a gas, for example such as methane or ethane.

In one embodiment the parameter of the niche parameter is the cultivation process, in particular the cultivation process of the microbiome sample on the basal medium, such as batch fermentation, fed-batch fermentation or continuous fermentation.

In one embodiment, the parameter is radiation. Radiation sources include UV radiation, X-rays, gamma rays and more generally, cosmic rays. These different types of ionizing radiation, in particular UV and gamma rays, can impact microbial cells via direct and indirect (e.g., the formation of reactive oxygen species) mechanisms. In particular, the radiation can be comprised between 1 Gy and 30 kGy.

In one embodiment, the niche parameter is a bacterial inhibitor such as an antibiotic or antibodies. In one embodiment, the basal medium comprises an inhibitor of bacterial niche advantage, in particular a dietary product, a human-produced inhibitor or a bacterial inhibitor.

In one embodiment, the niche parameter is a virus, in particular a bacteriophage.

In one embodiment, the niche parameter is a bacterium, in particular bacteria of the niche, particularly bacteria resistant to antibiotics, such as MRSA (Methicillin-resistant Staphylococcus aureus). For example, the addition of particular bacteria into the basal medium can help to the characterization of overall presence and distribution of bacteria in the niche. This can be useful for the identification of competitive bacterial populations.

The parameter can also be gradient of any of the above parameter, for example pH and oxygen gradient.

The method according to the invention comprises the comparison between a niche condition and a niche condition of reference. Then, the invention may comprise the setup of a reference niche condition, and the assessment of “test niche conditions” that differs from the reference condition by at least on parameter such as described above. This allows the comparison between test and reference niche conditions. In one aspect, the niche condition and the niche condition of reference may differ by only one parameter. In another aspect, the niche condition and the niche condition of reference may differ by more than one parameter, for instance by 2, 3, 4 or 5 parameters.

“Reference conditions”, “niche condition of reference” or “reference niche condition” refer to conditions (e.g., specific set of biological parameters) that establish a basis of differential comparison with other niche conditions (e.g., wherein one parameter of interest is changed) among microbiome samples.

Then a reference niche condition can be any of the above, e.g., a niche condition wherein a parameter such as chemical entity, substrate, pH, Oxidation-Reduction Potential (Redox), temperature, flow rate, humidity, atmosphere, radiation, retention time, incubation time, inhibitory factors, or promoting growth factors is particularly defined.

The man skilled in the art knows how to define the reference condition, depending on the microbiome sample or conditions of interest.

For example, a reference niche condition can be defined by a parameter such as pH. Particularly, the pH of the reference niche condition can be 7. Then, in such example “a niche condition that differs in at least one parameter from the reference niche condition” is a niche condition that differs at least for pH, for example having a pH of 5, 6, 8 or 9.

Then, in one embodiment, the method for establishing a microbiome niche map comprising the steps of:
a. Providing a microbiome sample distributed into at least two microbiome test samples,
b. Growing a first microbiome test sample of step (a) in a reference niche condition, wherein the parameter of the niche condition is a particular pH,
c. Determining an absolute or relative abundance of individual microbe population in the first microbiome test sample at the end of step (b),
d. Growing a second microbiome test sample of step (a) in a niche condition that differs from the pH of the reference niche condition,
e. Determining an absolute or relative abundance of individual microbe population in the second microbiome test sample at the end of step (d),
f. Determining microbe population differentially enriched between the first and the second test sample by subtracting the absolute or relative abundance of an individual microbe population of step c) from the absolute or relative abundance of the same individual microbe population of step e),
g. Optionally, repeating steps (b) to (f) for at least one other niche parameter, preferably such as chemical entity, substrate, Oxidation-Reduction Potential (Redox), temperature, flow rate, humidity, pressure, radiation, retention time, incubation time, inhibitory factors, and promoting growth factors.
h. Attributing the enriched microbe population of step (f) to a specific niche condition.

In a one aspect, the method uses the absolute abundance.

In an alternative aspect, the method uses the relative abundance.

For example, a reference niche condition can be defined by a parameter such as a substrate. Particularly, the substrate of the reference niche condition can be fibers. Then, in such example “a niche condition that differs in at least one parameter from the reference niche condition” is a niche condition that differs at least for the fiber used as substrate.

Then, in one embodiment, the method for establishing a microbiome niche map comprising the steps of:
a. Providing a microbiome sample distributed into at least two microbiome test samples,
b. Growing a first microbiome test sample of step (a) in a reference niche condition, wherein the parameter of the niche condition is a particular niche substrate, wherein the substrate is a fiber, preferably selected from the group consisting of pectin, arabinogalactan, beta-glucan, soluble starch, resistant starch, fructo-oligosacharides, galacto-oligosacharides, xylan, arabinoxylans and cellulose,
c. Determining an absolute or relative abundance of individual microbe population in the first microbiome test sample at the end of step (b),
d. Growing a second microbiome test sample of step (a) in a niche condition that differs from the substrate used in the reference niche condition,
e. Determining an absolute or relative abundance of individual microbe population in the second microbiome test sample at the end of step (d),
f. Determining microbe population differentially enriched between the first and the second test sample by subtracting the absolute or relative abundance of an individual microbe population of step c) from the absolute or relative abundance of the same individual microbe population of step e),
g. Optionally, repeating steps (b) to (f) for at least one other niche parameter, preferably such as chemical entity, pH, Oxidation-Reduction Potential (Redox), temperature, flow rate, humidity, pressure, radiation, retention time, incubation time, inhibitory factors, and promoting growth factors.
h. Attributing the enriched microbe population of step (f) to a specific niche condition.

In one aspect, the method uses the absolute abundance.

In an alternative aspect, the method uses the relative abundance.

The same reasoning can be applied for each of the parameters, in particular the parameters including chemical entity, substrate, pH, Oxidation-Reduction Potential (Redox), temperature, flow rate, humidity, pressure, radiation, retention time, incubation time, inhibitory factors, and promoting growth factors.

### Absolute and Relative Abundance

The method according to the invention particularly comprises a step of determining the absolute or relative abundance of an individual microbe population in the microbiome sample.

In one aspect, the method uses the absolute abundance.

In an alternative aspect, the method uses the relative abundance.

The abundance is the representation of a phylogenic unit in a particular ecosystem. It is usually measured as the number of individuals found per sample. The ratio of abundance of one phylogenic unit to one or multiple other phylogenic unit living in an ecosystem or niche is referred to as relative phylogenic unit abundances. Both indicators are relevant for computing biodiversity. Abundance is in simplest terms usually measured by identifying and counting every individual of every phylogenic unit in a given niche.

By “phylogenic unit” it is meant a microbe or a population of microbes of the same genotype, genus, family, species or strain, or of the same molecular origin.

A variety of methods are used to measure abundance and are known by the man skilled in the art.

Species abundance distribution (SAD) is one of the main uses of this measurement. SAD is a measurement of how common, or rare species are within a niche. This allows to assess how different species are distributed throughout a niche. SAD is one of the most basic measurements in ecology and is used very often, therefore many different methods of measurement and analysis have been developed and are known by the man skilled in the art.

Another example of this is Semi-Quantitative Abundance ratings. These are measurement methods which involve estimation based on viewing a specific area of a designated size. The two Semi-Quantitative Abundance ratings used are known as the D.A.F.O.R (D—phylogenic unit observed is “Dominant” in a given niche, A—phylogenic unit observed is “Abundant” in a given niche, F—phylogenic unit observed is “Frequent” in a given niche, O—phylogenic unit observed is “Occasional” in a given niche, R—phylogenic unit observed is “Rare” in a given area) and the A.C.F.O.R. (A— phylogenic unit observed is “Abundant” within the given niche, C—phylogenic unit observed is “Common” within the given niche, F—phylogenic unit observed is “Frequent” within the given niche, O—phylogenic unit observed is “Occasional” within the given niche, R—phylogenic unit observed is “Rare” within the given niche).

Abundance estimation also comprises statistical methods for estimating the number of individuals in a population.

An “individual microbiome population” is defined as a collection of microbes that share a common trait, such a trait can be of physiological, structural, or genetic nature, for example but not limited to, the same taxonomic unit (e.g. family, class, genus, or species), individual genes or gene clusters, motility, or gram-staining properties. In a particular aspect, by “individual microbe population”, it is meant a population of microorganism, preferably bacteria, that belongs to the same phylogenic unit.

The “absolute abundance” is defined as the individual microbe population size (eg. number of cells per volume) in the niche or in the microbiome.

In one embodiment, the absolute abundance of the individual microbe population is determined by (i) determining the total microbial growth at the end of the growing step (b) or (d), and (ii) determining the relative abundance of the individual microbe population grown at the end of the growing step (b) or (d), respectively. The absolute abundance is then calculated by multiplying the relative abundance of the individual microbe population by a quantity that is proportional to the bacterial growth, such as DNA concentration, number of gene copies per ml, CFUs or total cell counts.

Preferably, the absolute abundance of the individual microbe population is determined by (i) determining the total increase of microbial DNA or the optic density at the end of the growing step (b) or (d), and (ii) sequencing the total microbial DNA at the end of the growing step (b) or (d), respectively.

The “relative abundance” is a component of biodiversity and refers to how common or rare an individual microbe population is relative to other microbe populations in a defined niche. Relative abundance is preferably the percent composition of an individual microbe population relative to the total number of microbes in the niche. Relative phylogenic unit abundances tend to conform to specific patterns that are among the best-known and most-studied patterns in microbial ecology. Different populations in a community exist in relative proportions; this idea is known as relative abundance. Relative phylogenic unit abundance and phylogenic unit richness describe key elements of biodiversity. The relative abundance of an individual microbe population is calculated by measuring a proxy for abundance (e.g., a number of sequencing amplicon reads mapped to a gene, a genome coverage . . . ) and by dividing the measured quantity of each individual microbe population by the sum of the measured quantity across all the microbiome.

Absolute and/or relative abundance can be measured using optical density, qPCR, flow cytometry, chamber counting, total bacterial DNA quantification or metagenomic sequencing and grouping of genes. These methods are well known by the man skilled in the art.

In one embodiment, the abundance is measured by Amplicon sequence variant (ASV) techniques. ASV refers to individual DNA sequences recovered from a high-throughput marker gene analysis following the removal of spurious sequences generated during PCR amplification and sequencing. ASVs are thus inferred sequences of true biological origin. The term was introduced to distinguish between traditional methods that delineate operational taxonomic units (OTUs) generated by clustering sequences based on a shared similarity threshold and newer alternative methods that resolve individual sequences without clustering. The ASV approach is known to the person skilled in the art and will first determine which exact sequences were read and how many times each exact sequence was read. These data will be combined with an error model for the sequencing run. This will enable the comparison of similar reads to determine the probability that a given read at a given frequency is not simply due to a sequencing error. This creates a p-value for each exact sequence, where the null-hypothesis is equivalent to that exact sequence being a consequence of a sequencing error.

Sequences are then filtered according to some threshold value for confidence, leaving behind a collection of exact sequences with a defined statistical confidence. No clustering or reference databases were used, this is why ASV results can be readily compared between studies using the same target region.

Then, in one embodiment, the abundance is the abundance of an individual ASV.

The method according to the invention particularly comprises a step of determining microbe population differentially enriched between a first and a second test sample by subtracting the absolute abundance of an individual microbe population grown in the niche reference condition from the absolute abundance of the same individual microbe population in a test niche condition.

This step is of particular interest for the niche mapping because it allows to put the focus on the microbe population differentially enriched in the considered niche conditions. It is a means to discard the background noise.

Differential enrichment is of particular interest for the niche mapping. Microbe populations which grow equally well under niche conditions and under reference conditions are not specific for a niche. By looking for differentially enriched microbe populations, specific inhabitants of a niche can be identified.

The term “enrichment” or “enrichment culture” is the use of certain growth conditions to favor the growth of a particular microorganism over others, enriching a sample for the microorganism of interest. This is generally done by introducing nutrients or environmental conditions (i.e., niche conditions). Enrichment cultures are used to increase a small number of desired organisms to detectable levels.

By “specifically enriched” or “differentially enriched” it is meant that a microorganism, in particular a bacterium, grow differentially depending on the niche conditions. It can refer to differences in terms of biomass yield and/or growing rate between two or more niche conditions.

Alternatively, instead of determining an absolute abundance of individual microbe populations in step c) and e), it is possible to assess the absolute increase in abundance of individual microbe populations, by measuring the abundance before the growing step and after the growing step, thereby being able to determine in step f) the microbe differentially enriched by subtracting the absolute increase in abundance of an individual microbe population of step c) from the absolute increase in abundance of an individual microbe population of step e).

An increase is measured for the reference condition and for the niche conditions to be tested. The subtraction of the increase of reference condition to the increase of the test niche condition (e.g., increase in test condition—increase in reference condition) allows to evaluate enrichment.

Then, the steps (c) and (e) of the method may comprises determining an absolute increase of individual microbe population in the microbiome test sample between the beginning of step (b) and the end of step (b) or between the beginning of step (d) and the end of step (d), respectively.

Step (f) of the method may comprise determining microbe population increased between the first and the second test sample by subtracting the absolute increase of an individual microbe population of step c) from the absolute increase of the same individual microbe population of step e),

For example, when for the reference condition 10 (whatever unit) is measured before the growing step and 12 is measured after the growing step, the increase is 2. For the niche condition 10 is measure before the growing step and 20 after the growing step, then the increase is 10. Then, the enrichment is 8 (10−2).

This differences in terms of enrichment and bacterial growth between niche conditions allows to establish microbial niche and the niche conditions necessary for an individual microbe population or bacteria to grow. This defines particular pattern both in terms of niche conditions themselves but also provides insight of the niche conditions that individual microbe population and particular bacteria can colonize.

### Microbial Niche

The aim of the method according to the invention is to establish a niche map of a particular microbiome.

The conditions of the niche may be adapted to recreate an environment similar to which the microbiome is sampled. For example, for recreating gut conditions, the parameter of substrate, pH and oxygen can be optimized by the man skilled in the art.

In one embodiment, more than one niche parameter may vary to obtain a particular niche condition. In such embodiment, the method may comprise at least 1, 2, 3, 4, 5, 6, 7, 8, 9, 10 different parameters such as described above, for example for creating naturally occurring ecological niche.

The method according to the invention particularly comprises an iteration step.

Then, the invention comprises the repetition of steps (b) to (f) for at least one other niche conditions.

In particular, the repetition step is performed for covering at least 50%, 60%, 70%, 75%, 80%, 85%, 90%, 95% or 100% of the microbiome sample diversity or phylogenic unit richness.

“Phylogenic diversity” or “diversity” is the number of different phylogenic unit or individual microbe population that are represented in a given community, microbiome or niche. The effective number of phylogenic units refers to the number of equally abundant phylogenic unit needed to obtain the same mean proportional phylogenic unit abundance as that observed in the community, microbiome or niche of interest. Diversity may include phylogenic richness, taxonomic or phylogenetic diversity, and/or phylogenic evenness.

“Phylogenic richness” is the number of different phylogenic unit represented in a community, microbiome or niche. Phylogenic richness is simply a count of phylogenic unit, and it does not take into account the abundances of the phylogenic unit or their relative abundance distributions. Phylogenic richness is sometime considered synonymous with diversity, but the formal metric diversity takes into account both phylogenic richness and phylogenic evenness.

The repetition can be performed 0, 1, 2, 3, 4, 5, 6,7 ,8, 9, 10, 15, 20 times, or more. Preferably, the step (g) of the method is repeated several times, preferably at least two times, in which each time the niche parameters vary.

For example, when the first assessed parameter is pH, the other niche parameter can be selected from the group consisting of chemical entity, substrate, Oxidation-Reduction Potential (Redox), temperature, flow rate, humidity, pressure, radiation, retention time, incubation time, inhibitory factors, and promoting growth factors.

For example, when the first assessed parameter is substrate, the other niche parameter can be selected from the group consisting of chemical entity, pH, Oxidation-Reduction Potential (Redox), temperature, flow rate, humidity, pressure, radiation, retention time, incubation time, inhibitory factors, and promoting growth factors. Also, when the first assessed parameter is substrate, for example a fiber such as pectin, the other niche parameter can also be another particular substrate, for example such as another fiber than pectin, particularly arabinogalactan, beta-glucan, soluble starch, resistant starch, fructo-oligosacharides, galacto-oligosacharides, xylan, arabinoxylans or cellulose or another type of substrate such as a protein or a metabolic acid.

The method may also optionally comprise repeating step (b) to (g) for multiple microbiomes of the same or different type, or for a microbiome provided from different environments or subjects.

In particular, the method can be carried out for several microbiomes, for example microbiome samples from different subjects or population of subjects, preferably healthy subjects or subjects suffering from a disease or a disorder.

Finally, the method according to the invention comprises a step of attributing the enriched microbe of step (f) to a niche condition.

One phylogenic unit can be attributed to a single condition or to multiple niches conditions. This can particularly depend on the number of niche condition parameter that vary in the method and the number of repetition step.

### Uses

The niche map according to the invention can find many applications and uses. The fields of application are human and animal health as well as environmental fields of applications e.g., soil or sewage, agriculture, farming, and food industry.

For example, the niche mapping method according to the invention can be used for:
identifying a condition, the condition being (i) a dysbiosis, (ii) a presence of or a susceptibility to develop a disease, (iii) a susceptibility of a subject to be a responder or a non-responder to a treatment, for instance a treatment with a drug or diet, (iv) a susceptibility of a subject to present or not side effects to a treatment with a drug, or (v) a good health,
monitoring and prediction of nutritional interventions,
monitoring the effects of drugs on the microbiome,
monitoring effects of pollutants on the microbiome,
designing a strategy to overcome a dysbiosis,
developing adjuvant therapies for mitigating the adverse effects of a drug,
determining under-representation and/or over-representation of niches in clinical datasets compared to the niche map of healthy donors,
developing a composition of one or more microbial strains representing under-represented niche inhabitants, for example a probiotic composition susceptible to benefit to or for the treatment of a patient suffering from a disease or a disorder, for example such as a dysbiosis,
developing a nutritional intervention to induce a targeted change in the microbiome,
giving dietary recommendations to induce a change or an improvement of the microbiome,
monitoring changes in the microbiome and changes in physiological parameters such as inflammation, metabolism, glucose levels and the like,
predicting the response of a subject to a treatment, in particular with a drug or a diet,
investigating the taxonomic and metabolic diversity of a microbiome, for example the lactate and succinate utilizing bacterial communities present in microbiomes of healthy subject(s),
investigating novel or alternative source of carbon or nitrogen for a microbiome,
identifying novel bacterial population and/or bacterial strains.

The present invention can also be used to predict or monitor effects of nutritional interventions, drugs, or other substances on a microbiome. It can also be used to recommend strategies to treat microbiome disbalance.

Then the method according to the invention identifies a niche pattern or niche signature that allows to discriminate a feature, characteristic, condition or state. The niche pattern or niche signature indicative of a feature, characteristic, condition or state is based on enriched individual microbe population in a population of microbiomes having this feature, characteristic, condition or state in comparison to a controlled population of microbiomes having not this feature, characteristic, condition or state. In other words, the method identifies a pattern of enriched individual microbe populations associated to a feature, characteristic, condition or state.

An “under-represented” group (e.g., niche or niche inhabitants) describes a subset of a population that holds a smaller percentage or number within a significant subgroup than the subset holds in the general population. It particularly refers to a percentage or number of niche or niche inhabitants that is smaller in the tested conditions (for example a microbiome from a patient suffering from a disease) in comparison to a reference condition (for example a microbiome from a healthy patient), or to the absence of niche or niche inhabitants int the tested conditions compared to a reference condition.

An “over-represented” group (e.g., niche or niche inhabitants) describes a subset of a population that holds a higher percentage or number within a significant subgroup than the subset holds in the general population. It particularly refers to a percentage or number of niche or niche inhabitants that is higher in the tested conditions (for example a microbiome from a patient suffering from a disease) in comparison to a reference condition (for example a microbiome from a healthy patient).

Particularly, a niche pattern or signature can be typical of a healthy microbiome, subject or environment, and another niche pattern or signature can be typical of a dysfunction, a disbalance, a disorder, a disease, or pollution.

For example, the niche pattern can be determined by comparing:
a niche map of microbiome samples of healthy patient(s) and a niche map of microbiome samples of patient(s) suffering or susceptible to suffer from a disease, thereby identifying a niche pattern indicative of or associated to patient(s) suffering or susceptible to suffer from a disease;
a niche map of microbiome samples of responder(s) and a niche map of microbiome samples of a non-responder(s) to a treatment or a diet, thereby identifying a niche pattern indicative of or associated to the response or the absence of response to said treatment or diet;
a niche map of microbiome samples of patient(s) presenting side effects to a treatment and a niche map of microbiome samples of patient(s) that does not, thereby identifying a niche pattern indicative of or associated to occurrence of side effects to a treatment in a patient;
a niche map of microbiome samples of healthy environment(s) and a niche map of microbiome samples of polluted environment(s), thereby identifying a niche pattern indicative of or associated to healthy environment(s) or to polluted environment(s);
a niche map of microbiome samples of healthy or edible food and a niche map of microbiome samples of unsuitable food for consumption, thereby identifying a niche pattern indicative of or associated to the edible food or the food unsuitable for consumption.

Then, the method according to the invention allows the comparison of microbiomes, based on their niche map characteristics.

The invention also relates to a method for developing a composition for the treatment of a subject or environment(s) having a feature, characteristic, condition or state, wherein the method comprises:
(a) determining the niche map of the subject or environment(s) microbiome by the method according to the invention,
(b) comparing the microbiome niche map of the subject or environment(s) to a microbiome niche map of reference,
(c) determining under-representation and/or over-representation of niches in the subject or environment(s) microbiome compared to the niche map of reference, and
(d) developing a composition comprising one or more bacterial strains representing an underrepresented niche, and optionally one or more bacterial strains as stabilizing agents,
(e) optionally, administering the composition of step (d) in the subject or contacting the environment(s).

The invention also relates to a method for developing a probiotic composition susceptible to benefit to a patient suffering from dysbiosis, wherein the method comprises:
(a) determining the niche map of the subject microbiome by the method according to the invention,
(b) comparing the microbiome niche map of the subject to a microbiome niche map of a healthy subject or a healthy subject population,
(c) determining under-representation and/or over-representation of niches in the subject microbiome compared to the niche map of a healthy subject or a healthy subject population,
(d) developing a composition comprising one or more bacterial strains representing an underrepresented niche, and optionally one or more bacterial strains as stabilizing agents, and
(e) optionally, administering the composition of step (d) in the patient, optionally with a pharmaceutical acceptable carrier.

By bacterial strains as stabilizing agents it is intended to refer to bacterial strains that sustain or support the presence of another single or set of bacteria through direct or indirect interaction.

The invention also relates to a method for developing a composition for the treatment of a patient suffering from a disease or a disorder, preferably a dysbiosis or a disease or disorder associated with dysbiosis, wherein the method comprises:
(a) determining the niche map of the subject microbiome by the method according to the invention,
(b) comparing the microbiome niche map of the subject to a microbiome niche map of a healthy subject or a healthy subject population,
(c) determining under-representation and/or over-representation of niches in the subject microbiome compared to the niche map of a healthy subject or a healthy subject population, and
(d) developing a composition comprising one or more bacterial strains representing an underrepresented niche, and optionally one or more bacterial strains as stabilizing agents,
(e) optionally, administering the composition of step (d) in the patient, optionally with a pharmaceutical acceptable carrier.

In particular, in step (d) the type and origin of bacteria, particularly bacterial strains, can be selected according to the targeted level of complexity of the microbiome.

Thus, the method according to the invention may provide for compositions for use in the prophylaxis, treatment, prevention or delay of progression of a disease related to a microbiome disbalance or associated with microbiota dysbiosis.

In human health, it is generally accepted that dysbiosis originates from an ecological disbalance (e.g., based on trophism), characterized by disproportionate amounts or absence of bacteria strains in the microbiome of the patient which are essential for the establishment and/or maintenance of a healthy microbiome. In one embodiment, the method according to the invention may provide for compositions for use in the prophylaxis, treatment, prevention or delay of progression of a disease or disorder selected from intestinal infections, including gastro-intestinal cancer, colorectal cancer (CRC), auto-immune disease, infections such as caused by virus or bacteria, ulcers, gastroenteritis, Guillain-Barre syndrome, graft versus host disease (GvHD), gingivitis and nosocomial infection. In particular, the disease can be selected from Clostridium difficile infection (CDI), vancomycin resistant enterococci (VRE), post-infectious diarrhea, inflammatory bowel diseases (IBD), including ulcerative colitis (UC) and Crohn's disease (CD).

In a particular embodiment, the disease or disorder to be treated involves bacteria of the human microbiome, preferably the intestinal microbiome, such as inflammatory or auto-immune diseases, cancers, infections or brain disorders. Indeed, some bacteria of the gut microbiome, without triggering any infection, can secrete molecules that will induce and/or enhance inflammatory or auto-immune diseases or cancer development.

In one embodiment, the probiotic composition of step (d) can be used in combination with additional treatment or composition that help to treat the patient's disease or disorder. For example, such additional treatment can be an anti-inflammatory agent, one or more immuno-suppressive or anti-cancer agents. Such immuno-suppressive agents may be glucocorticoids, cytostatics or antibodies. Such anti-cancer agents may be chemotherapy or radiotherapy agents, for example drugs, hormones or antibodies.

Physiological data of the patient or subject (e.g., age, size, and weight) and the routes of administration have to be taken into account to determine the appropriate dosage, so as a therapeutically effective amount of the probiotic composition will be administered to the patient or subject.

The invention also relates to a method for predicting the response of a subject to a treatment, preferably with a drug or a diet, wherein the method comprises:
(a) determining the niche map of the subject microbiome by the method according to the invention,
(b) comparing the microbiome niche map of the subject to a microbiome niche map of reference, the reference being a microbiome niche map of a good-responder population to such treatment or diet and/or a microbiome niche map of a non-responder population to such treatment or diet, and
(c) determining under-representation and/or over-representation of niches in the subject microbiome compared to the niche map of reference, wherein under-representation or over-representation of niches in the subject microbiome being indicative of the response of the subject to such treatment or diet.

The invention also relates to a method for predicting the susceptibility of a subject to present side effects from a treatment, preferably with a drug or a diet, wherein the method comprises:
(a) determining the niche map of the subject microbiome by the method according to the invention,
(b) comparing the microbiome niche map of the subject to a microbiome niche map of reference, the reference being a microbiome niche map of a healthy patient or group of patients or of a patient or group of patients that follow the same treatment and do not present side effects, and
(c) determining under-representation and/or over-representation of niches in the subject microbiome compared to the niche map of reference, wherein under-representation or over-representation of niches in the subject microbiome being indicative of the susceptibility of the subject to have side effects from the treatment.

Preferably, the monitoring of the susceptibility of the subject is assessed through time, for example as long as the patient follows the treatment or even after the treatment has stopped, preferably every week, every month, every three months, every six months, every year, every five years or every 10 years.

The invention also relates to a method for monitoring the health of a subject, wherein the method comprises:
(a) determining the niche map of the subject microbiome by the method according to the invention,
(b) comparing the microbiome niche map of the subject to a microbiome niche map of reference, the reference being a microbiome niche map of a healthy subject or a population of healthy subjects, and
(c) determining under-representation and/or over-representation of niches in the subject microbiome compared to the niche map of reference, wherein the absence of under-representation or over-representation of niches in the subject microbiome is indicative of the health of the subject.

This monitoring method can additionally comprise measurement of a physiological parameter of a subject, such as inflammation markers, glucose blood concentration, glycemia, weight, body mass index (BMI) blood pressure, cholesterol, and any combination thereof.

Preferably, the monitoring of the health of the subject is assessed through time, for example, every week, every month, every three months, every six months, every year, every five years or every 10 years.

Further aspects and advantages of this invention are disclosed in the following experimental section, which should be regarded as illustrative and not limiting the scope of this application.

### EXAMPLES

### Example of the Steps of the Niche Mapping Method According to an Embodiment of the Invention

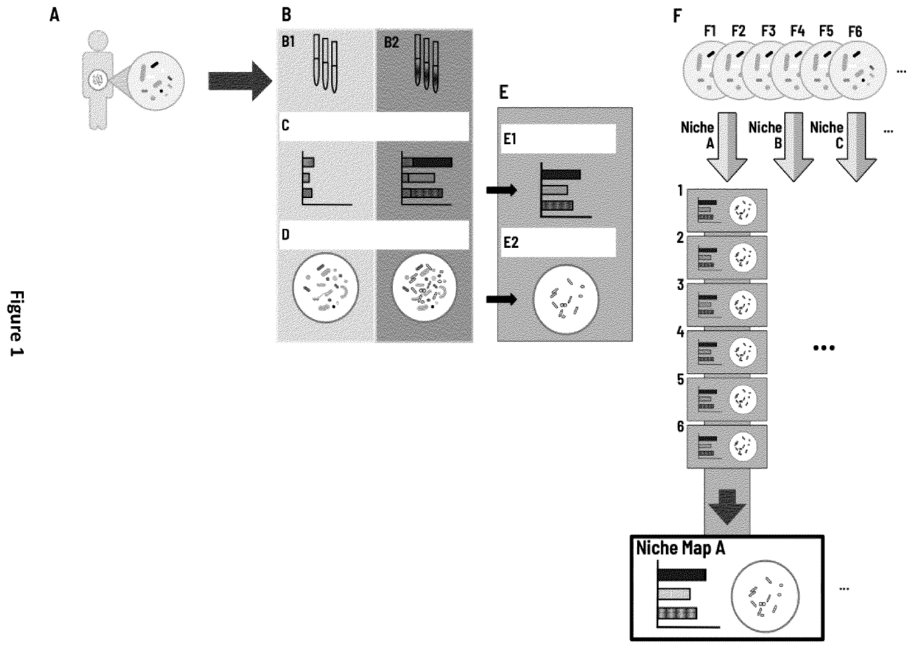

FIG. 1 shows an example of steps of the Niche Mapping method. Step A) Microbiome sample is obtained from a microbiome source (e.g., human or animal (intestine, lung, vaginal tract, skin, or other body part, etc.), agriculture (soil, plant surface, etc.), biotechnological process (wastewater, biofuel production, etc.), food (cheese, wine, beer, sourdough, etc.). Step B) The microbiome sample is grown in a reference condition B1 (background/noise) and in a specific niche condition B2 (foreground/signal) that differ by at least one parameter from the reference condition. Reference conditions are e.g., a basal medium without a specific niche substrate and the niche condition is e.g., the same basal medium with a specific niche substrate. Step C) The metabolic activity during and at the end of growth is measured, i.e., the production and consumption of metabolites. Step D) The compositional change is also measured, i.e., the absolute increase or decrease of microbial cells of each microbiome member during the enrichment. By comparing the differences in metabolic activity E1 and growth of cells between the specific and reference niche condition E2, the microbes that are most competitive for the specific niche in the particular microbiome sample can be determined. Step F) By repeating the process for many microbiome samples from different sources F1-F6, competitive microbes in each sample can be identified to map competitive bacteria to the specific niche.

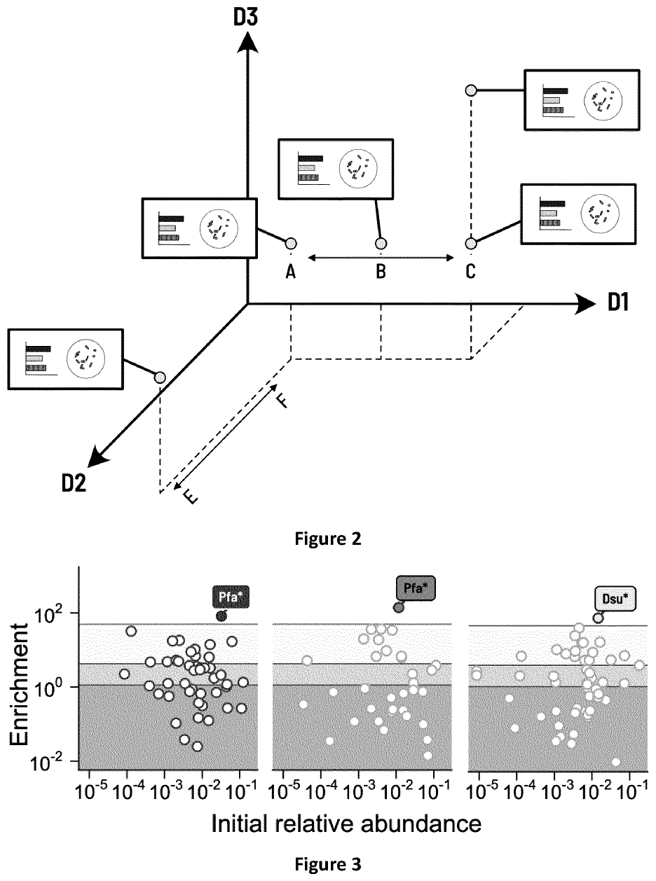

As illustrated by FIG. 2, Niches are mapped by varying the boundary conditions and the properties around the specific niches. For example, niches that differ in the carbon source are mapped by varying the carbon source of the niche substrate (D1 Axis i.e., by using substrate A, B, C). Niches that differ in physicochemical properties such as pH are mapped by varying the pH of the niche media (D2 Axis i.e., varying pH between E and F). Niches that differ in other physical properties, like growth rate are mapped by varying the incubation time (D3 Axis). The repetition of this niche mapping approach over sufficient microbiomes allows the prediction of most competitive bacteria in the specific niche.

### Succinate Niche

One particular type of niche is one where the main defining niche condition is a particular growth substrate of interest, for example succinate. In the context of the human intestine, microbial growth substrates are either directly supplied from the human food, or otherwise are breakdown products of the food. These breakdown products can be produced from host digestive activity, and can be metabolic products produced from microbial activity in the intestine. When other microbes consume these metabolic products as a growth substrate, this forms a ‘cross-feeding interaction’ between the two types of microbes. Succinate can be produced by a number of common intestinal bacteria. Because elevated levels of succinate have been associated with intestinal inflammation, mapping which bacteria occupy the ‘succinate niche’ is important.

In order to map the succinate niche, the inventors performed enrichments of microbiome samples in test growth conditions where the main carbon source was succinate. As a reference condition, they used the same growth media with the exception that the carbon source succinate was not added. They then measured which bacteria were enriched in the test condition (succinate niche conditions) relative to the reference condition. They computed the enrichment score for each amplicon sequence variant (ASV) as the log10-fold difference in abundance between the succinate condition and the reference condition, corrected for the initial relative abundance in the microbiome sample. They then ordered all ASVs by their corrected enrichment scores and marked the top 2.5% as putative members of the succinate niche. Using this procedure, they assigned the bacteria Phascolarctobacterium faecium and Dialister succinatiphilus to the succinate niche. In three example microbiome samples, two contained P. faecium and one contained D. succinatiphilus (FIG. 3). Both of these bacterial species are known succinate consumers, confirming that the method of the invention correctly assigns bacteria to the succinate niche.

### Lactate Niche

While some niches are primarily defined by the niche condition that identifies the growth substrate, additional niche conditions, like pH, can be important for microbial activity or microbial competition. These additional niche conditions thus further subdivide growth substrate niches, for example the lactate niche, into more specific ones, for example a ‘high pH lactate niche’ and a ‘low pH lactate niche’.

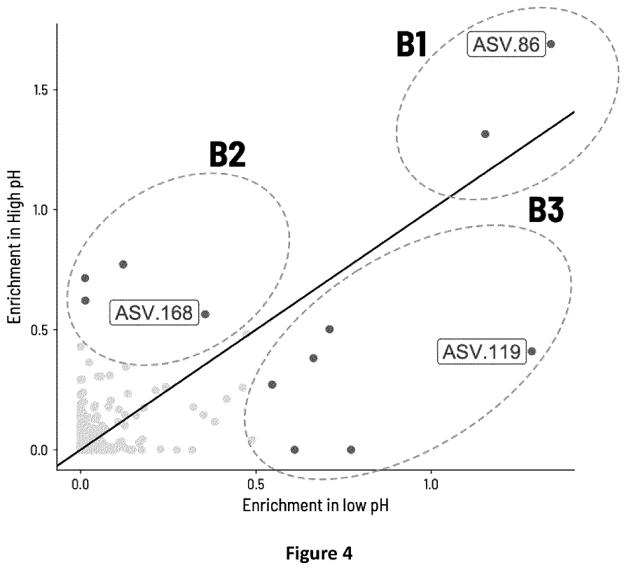

To map intestinal bacteria to the high and low pH lactate niches, the inventors performed enrichments in test conditions with lactate as the main carbon source and the pH adjusted to either 5.8 (low pH) and 6.5 (high pH), respectively. As reference conditions, they used basal growth media without the addition of lactate at either pH=5.8 or 6.5, respectively. They then determined which ASVs were specifically enriched in the low and high pH lactate conditions and assigned the ASVs to one of three categories (FIG. 4). Category B1 are ASVs that map to the lactate niche irrespective of pH. Category B2 are ASVs that are more strongly enriched in the high pH conditions than the low pH conditions, and thus map to the high pH lactate niche. Conversely, category B3 are ASVs that are more strongly enriched in the low pH conditions than the high pH conditions, and thus map to the low pH lactate niche. For example, the most enriched ASV of category B1 was taxonomically identified as Coprococcus_A species (ASV.86), ASV.168 mapped to category B2 and was taxonomically identified as an Anaerobutyricum species, and ASV.119 mapped to category B3 and was classified as an Anaerotignum species. All of these are known lactate-utilizing bacteria which confirms that the method of the invention can identify lactate utilizing species. But importantly, these can be further mapped to niches with more refined niche conditions, such as low and high pH. Taxonomy was determined based on the GTDB r89 nomenclature.

### Materials and Methods

### Collection of Feces.

Fresh fecal samples were donated from healthy individuals with no history of antibiotic use, intestinal infections, or severe diarrhea during the three months prior to making the donation. The donors did not take immunosuppressive drugs, blood thinners, or medication affecting the bowel passage or digestion. Fecal samples were anaerobically transported in an airtight container together with an Oxoid™ AnaeroGen™ 2.5 L sachet (Thermo Fisher Diagnostics AG, Pratteln, Switzerland) and processed within three hours after defecation. Stool consistency was evaluated optically according to the Bristol Stool Scale (Lewis and Heaton 1997) and samples within the defined range of a healthy stool, notably with a score between 3-5, were accepted.

### Culture Media and Anaerobic Dilution Solution.

Culture media were based on a common basal medium and supplemented with the growth substrate representative of the niche condition. All medium ingredients except sodium bicarbonate and L-cysteine HCl were dissolved in an Erlenmeyer flask and the pH was adjusted to pH=7 by titrating 5 mM sodium hydroxide. The media were boiled for 15 min for major removal of oxygen, under constant moderate stirring, and using a Liebig condenser to prevent vaporization of ingredients. After boiling, the media were constantly flushed with CO2. Sodium bicarbonate and L-cysteine hydrochloride monohydrate were added when the media cooled down to 55° C. for further reduction of residual oxygen for 10 min. Aliquots of 8 mL of medium were filled into Hungate tubes under constant flushing with CO2, and Hungate tubes were sealed with butyl rubber stoppers and screw caps (Millan SA, Geneva, Switzerland). The media were sterilized by autoclaving and subsequently stored at room temperature. Anaerobic dilution solution for fecal samples was prepared following the same procedure as for the culture media, except that aliquots of 9 mL were filled into Hungate tubes to facilitate serial dilutions.

### Feces Processing and Dilutions.

For processing, the fecal samples were transferred into a Coy anaerobic chamber (Coy Laboratories, Ann Arbor, Mich., USA) with an atmosphere of 10% CO2, 5% H2, and 85% N2. We prepared at 1:10 dilution with one gram of fecal sample that was measured with a sterile plastic spoon (VWR International, Dietikon, Switzerland) and subsequently suspended in 9 mL of anaerobic dilution solution in a sterile 50 mL Falcon tube using a 25 mL sterile serum pipet. We transferred 1 mL of the 1:10 dilution into a sterile 50 mL Falcon tube containing 9 mL of anaerobic dilution to obtain a 1:100 dilution. We then transferred 1 mL of the 10-2 dilution into a sterile Hungate tube containing 9 mL of anaerobic dilution solution. We performed subsequent serial dilutions in steps of 10 down to 10-11 outside of the anaerobic chamber under sterile, anaerobic conditions using the Hungate technique (Bryant 1972).

### Quantification of Total Viable Cells in Feces.

We estimated the total number of viable cells in feces by MPN enumeration in liquid culture and using strict anaerobic Hungate techniques (Sutton 2010). To this end, for each fecal sample we inoculated 0.3 mL of the 10-9, 10-10, and 10-11 dilutions into 8 mL of M2GSC growth medium in triplicates. We determined growth in each tube as an OD above 0.5. We performed a Bayesian estimation of the concentration of viable cells, μ, in the fecal samples by fitting a binomial model to the number of tubes for which we observed growth. We used a Gamma prior on μ with α=1 and β=0.01 and sampled from the posterior with RStan. All samples had viable cell numbers within a range of 1010-1012 cells per gram of feces, as expected for healthy stool (Franks et al. 1998).

In vitro Fermentations in Different Enrichment Conditions.

Anaerobic in vitro fermentations were performed in Hungate tubes sealed with butyl rubber stoppers and screw caps (Millan SA, Geneva, Switzerland). For each fermentation, 0.3 mL of the 10-8 fecal sample dilution was inoculated into 8 mL of cultivation medium under sterile and anaerobic conditions using the Hungate technique (Bryant 1972). All cultures were incubated at 37° C. After 48 h of incubation, we measured optical density at a wavelength of 600 nm directly in the Hungate tubes with a WPA CO 8000 Cell Density Meter (Biochrom Ltd, Cambridge, England), the metabolite profile, and microbial community composition.

### Microbial Metabolite Analysis.

Metabolite concentrations of SCFAs (formate, acetate, propionate, butyrate, and valerate), branched-chain fatty acids (BCFAs) (isobutyrate and isovalerate), and of intermediate metabolites (lactate, succinate, and ethanol) were measured by HPLC analysis. Samples were prepared from 1 mL of bacterial culture centrifuged at 14'000 g for 10 min at 4° C. The supernatant was filtered into 2 mL short thread vials with crimp caps (VWR International GmbH, Schlieren, Switzerland) using non-sterile 0.2 μm regenerated cellulose membrane filters (Phenomenex Inc., Aschaffenburg, Germany). A volume of 40 μL of sample was injected into the HPLC with a flow rate of 0.6 mL/min at a constant column temperature of 80° C. and using a mixture of H2SO4 (10 mM) and Na-azide (0.05 g/L) as eluent. Analyses were performed with a Hitachi Chromaster 5450 RI-Detector (VWR International GmbH, Schlieren, Switzerland) using a Rezex ROA-Organic Acid (4%) precolumn connected to a Rezex ROA-Organic Acid (8%) column, equipped with a Security Guard Carbo-H cartridge (4×3.0 mm). Metabolite concentrations were determined using external standards (all purchased from Sigma-Aldrich, Buchs, Switzerland) via comparison of the retention times. Peaks were integrated using the EZChromElite software (Version V3.3.2.SP2, Hitachi High Tech Science Corporation).

### DNA Extraction.

For fecal samples, we extracted total genomic DNA from 200 mg of each sample. For fermentations, we centrifuged 1 mL of bacterial cultures at 14'000 g and 4° C. for 10 mins. For both sample types, we used the FastDNA® SPIN Kit for Soil (MP Biomedicals, Illkirch Cedex, France) according to the manufacturer's instructions. We quantified the total DNA concentration using the Qubit® dsDNA HS Assay kit (Thermo Fisher Scientific, Pratteln, Switzerland).

Amplicon sequence variants and taxonomic assignment. We performed amplicon sequencing of the 16S rRNA V3-V4 region on the MiSeq platform (Illumina, Calif., USA) using the primer combination 341F (5″-CCTACGGGNBGCASCAG-3) and 806bR (5″- GGACTACNVGGGTWTCTAAT-3″). Library preparation and sequencing was performed by StarSEQ GmbH (Mainz, Germany) with 25% PhiX to balance the composition of bases. Amplicon Sequence Variants (ASVs) were inferred using Dada2 v1.18.0 with read length filtering set to c(250, 210), maxEE set to c(4,5), inference done in “pseudo pool” mode. Read pairs were merged with minOverlap of 20, and Bimeras were removed using the “consensus” method. The prepared GTDB r95 taxonomic database for Dada2 (GTDB ref, Dataset Ref) was used for taxonomic annotations via the assignTaxonomy function in Dada2.

### Computation of Abundance in Samples.

We computed the relative abundance of an ASV in a sample as the total number of sequencing reads that mapped to the ASV divided by the total number of sequencing reads in the sample. We computed the absolute abundance of an ASV as the total DNA in the sample multiplied by the relative abundance of the ASV.

### Computation of Enrichment Score in FIG. 3.

We first computed the logarithm of the difference in absolute abundance of an ASV between the samples enriched on basal medium with succinate and the corresponding samples enriched on the same basal medium without succinate. To correct for influences of the relative abundance in the corresponding fecal sample, we computed the enrichment score as the residuals of the regression of the logarithm of the difference in absolute abundance on the relative abundance in the fecal sample.

### Computation of Enrichment in FIG. 4.

We computed the enrichment as the difference in absolute abundance of an ASV between the samples enriched on the basal medium with lactate, once with pH=5.8 and once with pH=6.5, and the same sample enriched on the basal medium without lactate.

1-17. (canceled)

18. A method for establishing a microbiome niche map comprising the steps of:
(a) providing a microbiome sample distributed into at least two microbiome test samples,
(b) growing a first microbiome test sample of step (a) in a reference niche condition,
(c) determining an absolute or relative abundance of individual microbe population in the first microbiome test sample at the end of step (b),
(d) growing a second microbiome test sample of step (a) in a niche condition that differs in at least one parameter from the reference niche condition,
(e) determining an absolute or relative abundance of individual microbe population in the second microbiome test sample at the end of step (d),
(f) determining microbe population differentially enriched between the first and the second test sample by subtracting the absolute or relative abundance of an individual microbe population of step (c) from the absolute or relative abundance of the same individual microbe population of step (e),
(g) optionally, repeating steps (b) to (f) for at least one other niche condition, and
(h) attributing the enriched microbe population of step (f) or (g) to a specific niche condition.

19. The method according to claim 18, wherein the method uses the absolute abundance.

20. The method according to claim 18, wherein the method uses the relative abundance.

21. The method according to claim 18, wherein the absolute abundance of the individual microbe population is determined by (i) determining the total microbial growth at the end of the growing step (b) or (d), and (ii) determining the relative abundance of the individual microbe population grown at the end of the growing step (b) or (d), respectively.

22. The method according to claim 18, wherein the absolute abundance of the individual microbe population is determined by (i) determining the total increase of optical density or microbial DNA at the end of the growing step (b) or (d), and (ii) sequencing the total microbial DNA at the end of the growing step (b) or (d), respectively.

23. The method according to claim 18, wherein, in step (a), the method further comprises one or several dilutions of the microbiome sample in a suitable dilution agent.

24. The method according to claim 18, wherein, in step (a), the method further comprises a pre-treatment step of the microbiome sample, in particular using heat, pH stress, bleach or ethanol.

25. The method according to claim 18, wherein the step (g) is repeated at least two time with each time a different niche condition or parameter.

26. The method according to claim 18, wherein the niche condition parameter is selected from the group consisting of substrate, pH, Oxidation-Reduction Potential (Redox), temperature, humidity, pressure, cultivation method, incubation time, inhibitory factors and promoting growth factors.

27. The method according to claim 18, wherein the niche condition parameter is a niche substrate selected from the group consisting of carbohydrate, fiber, protein, gas, organic molecules of animal, fungi or plant origin, phenols, hormones, nucleotides and amino acids.

28. The method according to claim 27, wherein the niche substrate is selected from the group consisting of Cellulose, Hemicellulose, Guar Gum, Gum Arabic, Lignin, Fructan (long chain length), Inulin (long chain length); Arabinogalactan, Arabinoxylan, B-Glucan, Galactomannan, Glucomannan, Xyloglucan, Xylan, Amylo- pectin, Pectin, Starch (Type 1 to Type 9), Resistant starch (Type 1 to 3), Resistant dextrins, Arabinose, Fructose, Glucose, Galactose, Galacturonic Acid, Xylose, Lactose, Lactulose, Maltose, Sucrose, Galactooligosaccharides (GOS), Fructooligosaccharides (FOS), Xylooligosaccharides (XOS), Mannans, Pectin, Inulin, Polydextrose, Fungal Carbohydrates, Yeast carbohydrates, Chitin, Pullulan, Mucus, type I-type 4 mucus, N-acetyl-galactosamine, N-acetyl-glucosamine, Galactose, Fucose, human milk oligosaccharides, Siliac acid, N-Acetylneuraminic acid, Cell-surface glycans, GABA, surface glycosylation, Hormones, Cholesterol, Bile acids, yeast extract, casein, meat extract, blood, brain heart infusion broth, rumen fluid, sterile fecal suspension, amino acids, nucleic acids, biogenic amines, fetal calf serum, Acetate, Lactate, Formate, Succinate, H2, CO2, Ethanol, 1,2-Propanediol, and any combinations thereof.

29. The method according to claim 27, wherein the niche substrate is selected from the group consisting of polysaccharides, non-starch polysaccharides (NSP), resistant starch (RS) and oligosaccharides (RO) and any combination thereof.

30. The method according to claim 18, wherein the microbiome sample is provided from an intestinal microbiome, a mouth or nasal microbiome, a vaginal microbiome, a skin microbiome, a waste-treatment microbiome, a food microbiome, a microbiome used for food fermentation, oil spills microbiome, water microbiome, a microbiome from lakes, a soil microbiome or a plant-associated microbiome.

31. The method according to claim 18, wherein the method is carried out for microbiome samples from different subjects or population of subjects.

32. The method according to claim 18, wherein the microbiome sample is from a mammal.

33. The method according to claim 32, wherein said mammal is a human.

34. A method for identifying a pattern of enriched individual microbe populations associated with a condition in a subject, the condition being (i) a dysbiosis, (ii) a presence of or a susceptibility to develop a disease, (iii) a susceptibility of a subject to be a responder or a non-responder to a treatment, for instance a treatment with a drug or diet, (iv) a susceptibility of a subject to present or not side effects to a treatment with a drug, or (v) a good health comprising performing the method of claim 18 on a microbiome sample from the subject and identifying said condition.

35. A method for developing a probiotic composition susceptible to benefit to a patient suffering from dysbiosis, wherein the method comprises:
(a) determining the niche map of the subject microbiome by the method according to claim 18,
(b) comparing the microbiome niche map of the subject to a microbiome niche map of a healthy subject or a healthy subject population,
(c) determining under-representation and/or over-representation of niches in the subject microbiome compared to the niche map of a healthy subject or a healthy subject population, and
(d) developing a composition comprising one or more bacterial strains able to colonize an underrepresented niche and optionally, one or more bacterial strains as stabilizing agents.

36. A method for predicting the response of a subject to a treatment with a drug or a diet, wherein the method comprises:
(a) determining the niche map of the subject microbiome by the method according to claim 18,
(b) comparing the microbiome niche map of the subject to a microbiome niche map of reference, the reference being a microbiome niche map of a good-responder population to such treatment or diet and/or a microbiome niche map of a non-responder population to such treatment or diet, and
(c) determining under-representation and/or over-representation of niches in the subject microbiome compared to the niche map of reference, wherein under-representation or over-representation of niches in the subject microbiome being indicative of the response of the subject to such treatment or diet.
